(19) United States
(12) Patent Application Publication
(10) Pub. No.: US 2011/0224240 A1
(43) Pub. Date: Sep. 15, 2011

(54) METHODS OF TREATING CANCER AND RELATED DISEASES

(75) Inventors: Christopher M. PLEIMAN, Salt Lake City, UT (US); Gary G. MATHER, Salt Lake City, UT (US)

(73) Assignee: Myrexis, Inc., Salt Lake City, UT (US)

(21) Appl. No.: 13/004,656

(22) Filed: Jan. 11, 2011

Related U.S. Application Data
(60) Provisional application No. 61/294,034, filed on Jan. 11, 2010.

(51) Int. Cl. A61K 31/517; A61P 35/00; A61P 35/04

(52) U.S. Cl. 514/266.4

(57) ABSTRACT

Disclosed are orally bioavailable compounds effective as cytotoxic agents. The compounds of this invention are useful in the treatment of a variety of clinical conditions in which uncontrolled growth and spread of abnormal cells occurs, such as in cancer and related diseases.

### CROSS-REFERENCE TO A RELATED APPLICATION

This application claims the benefit of U.S. Provisional Application No. 61/294,034, filed Jan. 11, 2010, which is hereby incorporated by reference in its entirety.

### FIELD OF THE INVENTION

This invention is in the field of medicinal chemistry. In particular, the invention relates to methods of orally treating cancer and related diseases.

### BACKGROUND OF THE INVENTION

Cancer is a common cause of death in the world; about 10 million new cases occur each year, and cancer is responsible for 12% of deaths worldwide, making cancer the third leading cause of death globally. World Health Organization, National Cancer Control Programmes: Policies and Managerial Guidelines (2d ed. 2002).

Despite advances in the field of cancer treatment, the leading therapies to date include surgery, radiation, and chemotherapy. Chemotherapeutic approaches are said to fight cancers that are metastasized or that are particularly aggressive. Most of the cancer chemotherapy agents currently in clinical use are cytotoxins. Cytotoxic agents work by damaging or killing cells that exhibit rapid growth.

Accordingly, discovery of new and effective treatments for cancer is a high priority for health care researchers.

### BRIEF SUMMARY OF THE INVENTION

It was previously discovered that (2-aminomethylquinazolin-4-yl)-(4-methoxyphenyl)methylamine is a cytotoxic agent. Thus, (2-aminomethylquinazolin-4-yl)-(4-methoxyphenyl)methylamine is useful in treating or delaying the onset of diseases and disorders that are responsive to cytotoxic agents. It has now been discovered that (2-aminomethylquinazolin-4-yl)-(4-methoxyphenyl)methylamine is orally bioavailable in mammals, such as mice and primates.

In view of the above discovery, a first aspect of the invention includes a method of treating or ameliorating neoplasm or cancer comprising orally treating a warm-blooded animal, in need of such treatment, with an effective amount of the compound (2-aminomethylquinazolin-4-yl)-(4-methoxyphenyl)methylamine, or a pharmaceutically acceptable salt thereof.

A second aspect of the invention includes a method of treating an individual having cancer comprising orally administering to the individual a therapeutically effective amount of (2-aminomethylquinazolin-4-yl)-(4-methoxyphenyl)methylamine, sufficient to provide in the individual a plasma Cₘₐₓ of about 1 ng/mL to about 4,000 ng/mL, or an amount of a pharmaceutically acceptable salt of (2-aminomethylquinazolin-4-yl)-(4-methoxyphenyl)methylamine sufficient to achieve an equimolar concentration in the plasma of the individual.

A third aspect of the invention includes a method of treating an individual having cancer comprising orally administering to the individual a therapeutically effective amount of (2-aminomethylquinazolin-4-yl)-(4-methoxyphenyl)methylamine, sufficient to provide in the individual a plasma AUC_{(0 to ∞)} of about 1 hr*ng/mL to about 30,000 hr*ng/mL, or an amount of a pharmaceutically acceptable salt of (2-aminomethylquinazolin-4-yl)-(4-methoxyphenyl)methylamine sufficient to achieve an equimolar concentration in the plasma of the individual.

A fourth aspect of the invention includes a method of treating an individual having cancer comprising orally administering to the individual a therapeutically effective amount of (2-aminomethylquinazolin-4-yl)-(4-methoxyphenyl)methylamine, sufficient to provide in the individual a brain tissue AUC_{(0 to ∞)} of about 1 hr*ng/mL to about 210,000 hr*ng/mL, or an amount of a pharmaceutically acceptable salt of (2-aminomethylquinazolin-4-yl)-(4-methoxyphenyl)methylamine sufficient to achieve an equimolar concentration in the brain tissue of the individual.

A fifth aspect of the invention includes a method of treating an individual having cancer comprising treating the individual with about 20 mg/kg to about 200 mg/kg of (2-aminomethylquinazolin-4-yl)-(4-methoxyphenyl)methylamine, or an equimolar amount of a pharmaceutically acceptable salt thereof.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 depicts the extent of microtubule formation in A549 cells treated with (2-aminomethylquinazolin-4-yl)-(4-methoxyphenyl)methylamine (“Compound 1”). The cells in panel “a” were treated with 100 nM concentration of Compound 1. The cells in panel “b” were treated with 1 μM concentration of Compound 1. The cells in panel “c” were treated with 10 μM concentration of Compound 1.

FIG. 2 depicts the oral dose proportionality of (2-aminomethylquinazolin-4-yl)-(4-methoxyphenyl)methylamine to plasma Cₘₐₓ in mice.

FIG. 3 depicts the plasma and brain pharmacokinetic profile in mice resulting from a single per os dose of (2-aminomethylquinazolin-4-yl)-(4-methoxyphenyl)methylamine.

FIG. 4 depicts inhibition of B16-F0 tumor growth in allograft mice by (2-aminomethylquinazolin-4-yl)-(4-methoxyphenyl)methylamine. The inset depicts the change in average body weight for the allograft mice.

FIG. 5 depicts time to tumors reaching 1500 mm³ in the allograft mice referenced above.

FIG. 6 depicts a semilog plot of (2-aminomethylquinazolin-4-yl)-(4-methoxyphenyl)methylamine plasma concentration in a male Cynomolgus monkey after a single oral dose.

### DETAILED DESCRIPTION OF THE INVENTION

U.S. Pat. No. 7,618,975, issued Nov. 17, 2009, which is hereby incorporated by reference in its entirety, discloses numerous cytotoxic agents. In particular, U.S. Pat. No. 7,618,975 discloses (4-Methoxy-phenyl)-methyl-(2-methyl-quinazolin-4-yl)-amine hydrochloride, also known as MPC-6827 (Azixa™). MPC-6827 is a potent inducer of apoptosis that binds at or near the colchicine binding site on beta-tubulin and prevents the polymerization of tubulin into microtubules. MPC-6827 is an investigational new drug that has shown anti-cancer activity in clinical trials. However, it would be desirable to have an orally-bioavailable homolog of MPC-6827.

International Patent Application No. PCT/US09/50036, filed Jul. 9, 2009, which is incorporated by reference herein in its entirety, discloses that (2-aminomethylquinazolin-4-yl)-(4-methoxyphenyl)methylamine is a cytotoxic agent. Beyond that disclosed in International Patent Application No. PCT/US09/50036, it has been discovered that (2-aminomethylquinazolin-4-yl)-(4-methoxyphenyl)methylamine (also referred to herein as “Compound 1”) displays pro-apoptotic activity, with potency at low nanomolar concentrations in multiple cancer types including pancreatic, breast, colorectal, non-small cell lung, melanoma, ovarian cancers and leukemia. It has also been discovered that Compound 1 is equipotent for induction of apoptosis in multiple cancer cell lines, regardless of the expression levels for the multidrug resistance ATP-binding cassette (“ABC”) transporters MDR-1 (Pgp-1), MRP-1, and BCRP-1.

Importantly, it has further been discovered that Compound 1 has oral bioavailability that approaches 100% at a 60 mg/kg dose in mice with a half life (t_{1/2}) of approximately 10 hours.

Additionally, it has been discovered that Compound 1 crosses the blood brain barrier and distributes rapidly into the CNS, resulting in an exposure in the brain approximately 7 times higher than that in the plasma after oral or intravenous dosing.

Furthermore, it has been discovered that Compound 1 results in a statistically significant (p<0.00005) inhibition of tumor growth in melanoma (B16-F0) allografts in nude mice orally dosed with oral Compound 1.

In summary, it has been discovered that Compound 1 has potent and broad spectrum in vitro and in vivo antitumor activity with high oral bioavailability.

In view of the above discoveries, a first aspect of the invention includes a method of treating or ameliorating neoplasm or cancer comprising orally treating a warm-blooded animal, in need of such treatment, with an effective amount of the compound (2-aminomethylquinazolin-4-yl)-(4-methoxyphenyl)methylamine, or a pharmaceutically acceptable salt thereof. In some embodiments of this aspect, the warm-blooded animal is a human.

A second aspect of the invention includes a method of treating an individual having cancer comprising orally administering to the individual a therapeutically effective amount of (2-aminomethylquinazolin-4-yl)-(4-methoxyphenyl)methylamine, sufficient to provide in the individual a plasma Cₘₐₓ of about 1 ng/mL to about 4,000 ng/mL, or an amount of a pharmaceutically acceptable salt of (2-aminomethylquinazolin-4-yl)-(4-methoxyphenyl)methylamine sufficient to achieve an equimolar concentration in the plasma of the individual. In some embodiments of this method, the plasma Cₘₐₓ is between about 150 ng/mL and about 1,100 ng/mL.

A third aspect of the invention includes a method of treating an individual having cancer comprising orally administering to the individual a therapeutically effective amount of (2-aminomethylquinazolin-4-yl)-(4-methoxyphenyl)methylamine, sufficient to provide in the individual a plasma AUC_{(0 to ∞)} of about 1 hr*ng/mL to about 30,000 hr*ng/mL, or an amount of a pharmaceutically acceptable salt of (2-aminomethylquinazolin-4-yl)-(4-methoxyphenyl)methylamine sufficient to achieve an equimolar concentration in the plasma of the individual. In some embodiments of this aspect, the plasma AUC_{(0 to ∞)} is between about 8,000 hr*ng/mL to about 30,000 hr*ng/mL.

A fourth aspect of the invention includes a method of treating an individual having cancer comprising orally administering to the individual a therapeutically effective amount of (2-aminomethylquinazolin-4-yl)-(4-methoxyphenyl)methylamine, sufficient to provide in the individual a brain tissue AUC_{(0 to ∞)} of about 1 hr*ng/mL to about 210,000 hr*ng/mL, or an amount of a pharmaceutically acceptable salt of (2-aminomethylquinazolin-4-yl)-(4-methoxyphenyl)methylamine sufficient to achieve an equimolar concentration in the brain tissue of the individual. In some embodiments of this aspect, the brain tissue AUC_{(0 to ∞)} is between about 56,000 hr*ng/mL to about 210,000 hr*ng/mL.

A fifth aspect of the invention includes a method of treating an individual having cancer comprising treating the individual with about 20 mg to about 200 mg of (2-aminomethylquinazolin-4-yl)-(4-methoxyphenyl)methylamine per kilogram of bodyweight, or an equimolar amount of a pharmaceutically acceptable salt thereof per kilogram of bodyweight. In some embodiments of this aspect, treating the individual comprises orally administering (2-aminomethylquinazolin-4-yl)-(4-methoxyphenyl)methylamine to the individual. In some embodiments of this aspect, the method comprises treating the individual with about 50 mg to about 200 mg of (2-aminomethylquinazolin-4-yl)-(4-methoxyphenyl)methylamine per kilogram of bodyweight, or an equimolar amount of a pharmaceutically acceptable salt thereof per kilogram of bodyweight.

In some embodiments of each of the above aspects of the invention, the method further comprises administering at least one cancer chemotherapeutic agent other than Compound 1, or a pharmaceutically acceptable salt of the at least one cancer chemotherapeutic agent. In some embodiments, the at least one cancer chemotherapeutic agent is selected from alkylating agents, antimitotic agents, topoisomerase I inhibitors, topoisomerase II inhibitors, RNA/DNA antimetabolites, DNA antimetabolites, EGFR inhibitors, proteosome inhibitors, antibodies, and combinations thereof.

In some embodiments of each of the above aspects of the invention, the method further comprises administering radiation therapy at the same time or at a different time as treating with Compound 1.

In some embodiments of each of the above aspects of the invention, treating the warm-blooded animal with cancer, or the individual having cancer, further comprises surgically removing the cancer and then administering an effective amount of Compound 1 to the warm-blooded animal or individual.

In some embodiments of each of the above aspects of the invention, the cancer comprises a drug-resistant cancer. In some embodiments, the cancer comprises a primary cancer. In some embodiments, the cancer comprises a metastatic cancer and cancers that have metastasized from the site/location at which they originally appeared.

The above various methods of the present invention can be practiced by, or comprise, treating a warm-blooded animal, particularly a mammal, more particularly a human, with an effective amount of a compound according to the present invention. As used herein, the phrase “treating . . . with . . . a compound” means either administering the compound to an animal, or administering to an animal, the compound itself, or another agent to cause the presence or formation of the compound inside the cells or the animal.

It should be noted that in Compound 1, reference to any bound hydrogen atom can also encompass a deuterium atom bound at the same position. Substitution of hydrogen atoms with deuterium atoms is conventional in the art. See, e.g., U.S. Pat. Nos. 5,149,820 & 7,317,039. Such deuteration sometimes results in a compound that is functionally indistinct from its hydrogenated counterpart, but occasionally results in a compound having beneficial changes in its properties relative to the non-deuterated form. For example, in certain instances, replacement of specific bound hydrogen atoms with deuterium atoms dramatically slows the catabolism of the deuterated compound, relative to the non-deuterated compound, such that the deuterated compound exhibit a significantly longer half-life in the bodies of individuals administered such compounds. This is particularly the case when the catabolism of the hydrogenated compound is mediated by cytochrome P450 systems. Kushner et al., Can. J. Physiol. Pharmacol. (1999) 77:79-88.

Compound 1 may be prepared using methods known to those skilled in the art, or those disclosed in International Patent Application No. PCT/US09/50036, filed Jul. 9, 2009, which is incorporated by reference herein in its entirety. In one embodiment, the compound (2-aminomethylquinazolin-4-yl)-(4-methoxyphenyl)methylamine, or a pharmaceutically acceptable salt thereof, is prepared by a method comprising reacting 4-chloro-2-chloromethylquinazoline, or a salt thereof, with (4-methoxyphenyl)methylamine, or a salt thereof, under suitable conditions and with suitable reagents to form a first intermediate, (2-chloromethylquinazolin-4-yl)(4-methoxyphenyl)methylamine, or a salt thereof. The method further comprises reacting the first intermediate with a phthalimide salt under suitable conditions and with suitable reagents to form a second intermediate, 2-{4-[(4-methoxyphenyl)methylamino]quinazolin-2-ylmethyl}isoindole-1,3-dione, or a salt thereof. The method further comprises reacting the second intermediate with an amine base under suitable conditions and with suitable reagents to form (2-aminomethylquinazolin-4-yl)-(4-methoxyphenyl)methylamine, or a pharmaceutically acceptable salt thereof.

In one embodiment, the compound (2-aminomethylquinazolin-4-yl)-(4-methoxyphenyl)methylamine, or a pharmaceutically acceptable salt thereof, is prepared by a method comprising reacting 2,4-dichloro-quinazoline, or a salt thereof, and 4-methoxy-N-methylaniline, or a salt thereof, under suitable conditions and with suitable reagents to form a first intermediate, (2-chloroquinazolin-4-yl)(4-methoxyphenyl)methylamine, or a salt thereof. The method further comprises reacting the first intermediate with a cyanide salt under suitable conditions and with suitable reagents to form a second intermediate, 4-[(4-methoxyphenyl)-methylamino]-quinazoline-2-carbonitrile, or a salt thereof. The method further comprises reducing the carbonitrile moiety of the second intermediate under suitable conditions and with suitable reagents to form (2-aminomethylquinazolin-4-yl)-(4-methoxyphenyl)methylamine, or a pharmaceutically acceptable salt thereof.

In other embodiments, (2-aminomethylquinazolin-4-yl)-(4-methoxyphenyl)methylamine can be prepared as illustrated by the exemplary reactions in Schemes 1-2, below.

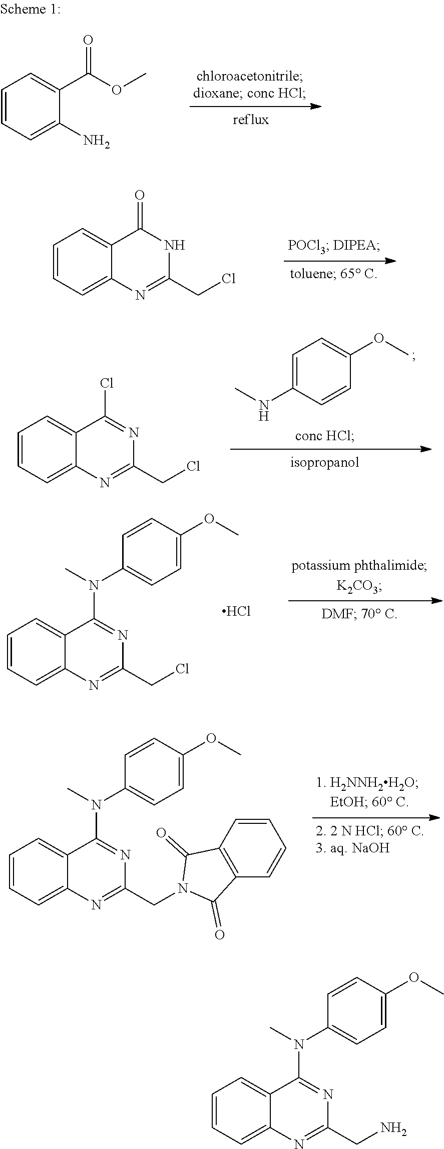

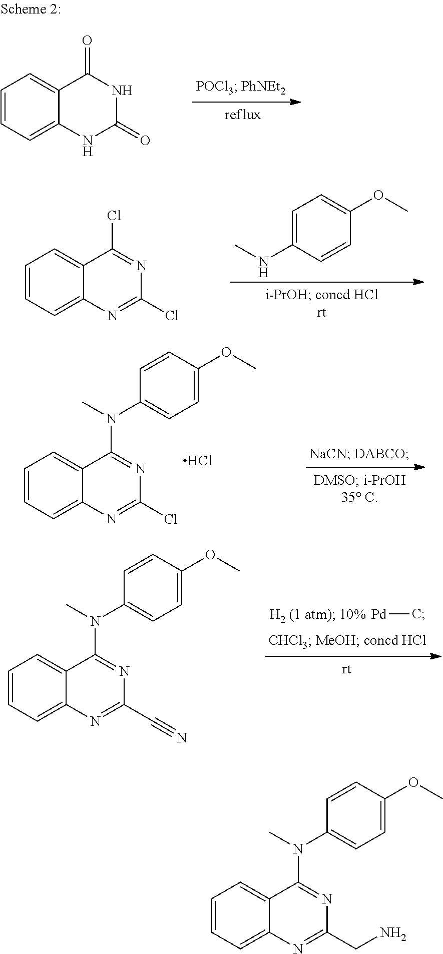

General: ¹H NMR were recorded at 400 MHz. HPLC were run on silica cartridges supplied by Teledyne Isco. Preparative TLC plates are Silica Gel GF 1000 μm 20×20 cm (Analtech 02013). Preparative RPLC were typically run on C₁₈ columns using a gradient of (0.01% TFA in acetonitrile) against (0.01% TFA in water).

### Scheme 1:

2-Chloromethyl-3H-quinazolin-4-one: A solution of methyl anthranilate (10.0 mL, 77.3 mmol) and chloroacetonitrile (5.5 mL, 87.1 mmol) in dioxane (150 mL) was treated with HCl (12 N, 10 mL, 120 mmol) and the resulting suspension refluxed overnight. The suspension was cooled to room temperature (rt) and the solid collected via vacuum filtration and washed with hexanes. The solid thus obtained was suspended in H₂O and neutralized with NaHCO₃. The solid was collected via vacuum filtration and dried under vacuum to yield 8.864 g (59%) of the title compound as a white solid. ¹H NMR (DMSO-d₆) δ 12.6 (s (br), 1H), 8.13 (ddd, 1H), 7.85 (ddd, 1H), 7.69 (ddd, 1H), 7.56 (ddd, 1H), 4.56 (s, 2H); LC-MS (ESI⁺; 195 ([M+H]+)).

4-Chloro-2-chloromethylquinazoline: A suspension of 2-chloromethyl-3H-quinazolin-4-one (12.27 g) in toluene (200 mL) was treated with Hünig's base (19 mL, 109 mmol) and POCl₃ (8.8 mL, 96.1 mmol) and heated to 65° C. overnight. The reaction was cooled to rt and the layers separated. The bottom layer was extracted with toluene. The top layers were combined and washed with cold H₂O and sat. NaHCO₃, dried (MgSO₄), filtered and concentrated. Purification by gradient MPLC (SiO₂, 120 g column, EtOAc/hexanes, 0-100%) provided 9.72 g (69%) of the title compound as a white solid. ¹H NMR (DMSO-d₆) δ 8.33 (ddd, 1H), 8.05-8.22 (m, 2H), 7.93 (ddd, 1H), 4.97 (s, 2H); LC-MS (ESI⁺; 213 ([M+H]⁺)).

(2-Chloromethylquinazolin-4-yl)(4-methoxyphenyl)methylamine hydrochloride: A suspension of 4-chloro-2-chloromethylquinazoline (7.383 g, 35.0 mmol) and (4-methoxyphenyl)methylamine (4.837 g, 35.3 mmol) in i-PrOH (50 mL) was treated with HCl (12 M, 1.5 mL, 18 mmol) and stirred at rt for 2 h. The resulting solid was collected by vacuum filtration, yielding 10.367 g (85%) of the title compound. ¹H NMR (DMSO-d₆) δ 7.80-7.94 (m, 2H), 7.40-7.80 (m, 2H), 7.26-7.34 (m, 1H), 7.07-7.15 (m, 2H), 6.83 (br d, 1H), 4.94 (s, 2H), 3.83 (s, 3H), 3.72 (s, 3H); LC-MS (ESI⁺; 314 ([M+H]⁺)).

2-{4-[(4-Methoxyphenyl)methylamino]quinazolin-2-ylmethyl}isoindole-1,3-dione: A suspension of (2-chloromethylquinazolin-4-yl)(4-methoxyphenyl)methylamine hydrochloride (10.367 g, 16.2 mmol) and K₂CO₃ (2.25 g, 16.3 mmol) in DMF (50 mL) was heated to 70° C. for 1 h. The reaction was cooled to rt, potassium phthalimide (6.004 g, 32.5 mmol) was added and the reaction heated to 70° C. for 2 h. The reaction was cooled to rt, diluted with EtOAc, washed with H₂O and 5% NaOH, dried (MgSO₄), filtered and concentrated. The residue was purified by gradient MPLC (SiO₂, EtOAc/hexanes 0-100%) to yield 8.56 g (68%) of the title compound. ¹H NMR (DMSO-d₆) δ 7.95-8.02 (m, 2H), 7.87-7.94 (m, 2H), 7.55-7.60 (m, 2H), 7.18-7.22 (m, 2H), 7.02-7.12 (m, 1H), 6.94-7.01 (m, 2H), 6.88 (dt, 1H), 4.95 (s, 2H), 3.77 (s, 3H), 3.26 (s, 3H). HRMS (ES) calcd for C₂₅H₂₁N₄O₃ (M+H) 425.1608, found 425.1604.

2-(Aminomethyl)-N-(4-methoxyphenyl)-N-methylquinazolin-4-amine: A solution of 2-{4-[(4-methoxyphenyl)methylamino]quinazolin-2-ylmethyl}isoindole-1,3-dione (8.561 g, 20.2 mmol) in EtOH (100 mL) was treated with hydrazine mono-hydrate (3.0 mL, 61.8 mmol) and heated to 60° C. for 2 h. The reaction was cooled to rt, HCl (2 N, 50 mL) added and the reaction heated to 60° C. for 30 min. After cooling to rt the solid was filtered off. The filtrate was concentrated, basified with 5% NaOH and extracted with CH₂Cl₂. The organic layers were combined, dried (MgSO₄), filtered and concentrated. The residue was purified by gradient reverse phase MPLC (MeCN/H₂O with 0.1% TFA) and the free base re-extracted as above to yield 3.10 g (52%) of the title compound. ¹H NMR (CDCl₃) δ 7.76 (d, 1H), 7.54 (ddd, 1H), 7.08-7.16 (m, 2H), 6.95-7.05 (m, 2H), 6.86-6.94 (m, 2H), 4.07 (s, 2H), 3.84 (s, 3H), 3.60 (s, 3H), 2.00 (s (br), 2H). ¹³C NMR (CDCl₃) δ 165.9, 162.0, 158.2, 152.2, 141.6, 132.0, 128.2, 127.6, 126.5, 124.4, 115.5, 115.4, 55.7, 49.1, 43.0. HRMS (ES) calcd for C₁₇H₁₈N₄O (M+H) 295.1553, found 295.1506.

### Scheme 2:

2,4-Dichloro-quinazoline: A suspension of 1H-quinazoline-2,4-dione (10 g, 62 mmol), POCl₃ (50 mL, 546 mmol) and N,N-dimethylaniline (1 mL, 7.9 mmol) was heated to reflux for 18 h. The reaction mixture was cooled to room temperature and poured slowly onto ice and extracted with CH₂Cl₂. The combined extracts were filtered through Na₂SO₄ and concentrated to give 4.2 g (34%) of 2,4-dichloro-quinazoline as a white solid.

(2-Chloro-quinazolin-4-yl)-(4-methoxy-phenyl)-methyl-amine hydrochloride: A solution of 2,4-dichloro-quinazoline (1 g, 5 mmol) and 4-methoxy-N-methylaniline (0.823 g, 6 mmol) in i-PrOH (17 mL) with HCl (12 M, 10 drops) was stirred at room temperature overnight. The reaction was filtered, washed with i-PrOH and dried under vacuum to provide 1 g (66%) of the title compound as a white solid. ¹H NMR (CDCl₃) δ 8.65 (d, 1H), 7.7 (t, 1H), 7.23 (d, 2H), 7.18 (t, 1H), 7.08 (d, 2H), 6.75 (d, 1H), 3.9 (s, 3H) 3.8 (s, 3H).

4-[(4-Methoxyphenyl)(methyl)amino]quinazoline-2-carbonitrile: To a solution of (2-chloroquinazolin-4-yl)-(4-methoxyphenyl)-methylamine hydrochloride (56 mg, 0.17 mmol) in 1 mL of DMSO and 0.5 mL of 2-propanol were added sodium cyanide (16 mg, 0.34 mmol) and 1,4-diazabicyclo[2.2.2]octane (9 mg, 0.084 mmol). The mixture was heated at 35° C. for 1 day with stirring. The solution was diluted with 5 mL of water and 10 mL of ethyl acetate. The organic layer was washed with water, then dried and concentrated. The resulting crude material was purified by silica gel column chromatography to give the title compound (34 mg, 70%). ¹H NMR (CDCl₃) δ 7.85 (d, 1H, J=8.4 Hz), 7.64 (t, 1H, J=6.8 Hz), 7.14 (m, 3H), 6.96 (m, 3H), 3.86 (s, 3H), 3.61 (s, 3H); LC-MS (ESI⁺; 291 ([M+H]⁺)).

(2-aminomethylquinazolin-4-yl)-(4-methoxyphenyl)methylamine: A mixture of 4-[(4-methoxyphenyl)-methylamino]-quinazoline-2-carbonitrile (22 mg, 0.076 mmol), 10% palladium on carbon (11 mg), concd HCl (0.05 mL) in 1 mL of chloroform and 5 mL of methanol was stirred under hydrogen (1 atm) overnight. The solution was passed through a pad of Celite and then concentrated. The resulting crude material was purified by MPLC (SiO₂/gradient of (1:1:8 Et₃N:MeOH:EtOAc) in EtOAc) to give the title compound (20 mg, 90%). ¹H NMR (CDCl₃) δ 7.74 (d, 1H, J=8.4 Hz), 7.53 (t, 1H, J=6.8 Hz), 7.11 (d, 2H, J=8.8 Hz), 7.00 (m, 2H), 6.90 (d, 2H, J=8.8 Hz), 4.06 (s, 2H), 3.83 (s, 3H), 3.59 (s, 3H); LC-MS (ESI⁺; 295 ([M+H]⁺)).

(2-aminomethylquinazolin-4-yl)-(4-methoxyphenyl)methylamine is useful in orally treating diseases that are responsive to cytotoxic agents. For example, (2-aminomethylquinazolin-4-yl)-(4-methoxyphenyl)methylamine is useful in the oral treatment of a variety of clinical conditions in which there is uncontrolled cell growth and spread of abnormal cells, such as in the case of neoplasia or cancer. Examples of such cancers include, but are not limited to, such specific diseases as Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma. Importantly, (2-aminomethylquinazolin-4-yl)-(4-methoxyphenyl)methylamine can be used in the treatment of primary cancers, or in the treatment of metastatic cancers, such as metastatic brain cancer.

Thus, the present invention includes therapeutic methods for the oral treatment of a variety of cancer types, comprising orally administering to an animal in need of such treatment an effective amount of (2-aminomethylquinazolin-4-yl)-(4-methoxyphenyl)methylamine, or a pharmaceutically acceptable salt thereof, wherein the therapeutic method is useful to treat the cancer present. Such cancers being a group of diseases characterized by the uncontrolled growth and spread of abnormal cells.

In practicing the therapeutic methods of the invention, effective amounts of compositions containing therapeutically effective concentrations of Compound 1, or a pharmaceutically acceptable salt thereof, formulated for oral application, for the treatment of neoplastic diseases and other diseases, including a variety of cancers, are orally administered to an individual exhibiting the symptoms of one or more of these neoplastic diseases. The effective amounts are effective in reducing, ameliorating, or eliminating one or more symptoms of the neoplastic disease. An effective amount of a compound for treating a particular disease is an amount that is sufficient to ameliorate, or in some manner reduce, the symptoms associated with that disease. Such an effective amount may be administered as a single dose or may be administered according to a dosage regimen, chosen for enhanced effectiveness. The effective amount of (2-aminomethylquinazolin-4-yl)-(4-methoxyphenyl)methylamine, or pharmaceutically acceptable salt thereof, may cure the disease but, typically, is administered in order to ameliorate the symptoms of the disease. Often, repeated administration is required to achieve the desired amelioration of symptoms or cure of the disease.

In practicing the methods of the present invention, in some embodiments, Compound 1, or a pharmaceutically acceptable salt thereof, and at least one known cancer chemotherapeutic agent are administered substantially simultaneously, i.e. the compounds are administered at the same time or one after the other, so long as the compounds reach therapeutic levels in the blood or brain at the same time. In another embodiment, the compound of the invention and at least one known cancer chemotherapeutic agent are administered according to their individual dosing schedules, so long as the compounds ultimately reach therapeutic levels in the blood or brain.

If a known cancer chemotherapeutic agent is also administered with Compound 1, or a pharmaceutically acceptable salt thereof, it is administered in an amount that is effective to achieve its intended purpose. The amounts of such known cancer chemotherapeutic agents effective for cancer treatment are well known to those skilled in the art. It should be noted however, that synergistic interactions between Compound 1, or a pharmaceutically acceptable salt thereof, and the known cancer chemotherapeutic agent may allow for the effective treatment of cancers with reduced dosages of one or both agents.

Multidrug resistance (MDR) is a major cause of cancer chemotherapy failure. Drug resistance is typically caused by ATP-dependent efflux of drug from cells by ATP-binding cassette (ABC) transporters. In particular, the ABC transporters ABCB1 (MDR-1, P glycoprotein); ABCC1 (MRP1); and ABCG2 (BCRP, MXR) are typically over-expressed in drug resistant tumors and thus are implicated in drug resistance. In comparison to most standard anti-cancer drugs, which are not effective in killing drug resistant cancer cells, Compound 1 is effective in killing drug resistant cancer cells. Therefore, Compound 1 or pharmaceutically acceptable salts thereof are useful for the oral treatment of drug resistant cancer.

Thus, one aspect of the present invention is the application of the methods as described above to orally treat or ameliorate tumors that have acquired resistance to other anticancer drugs. In one embodiment of this aspect of the invention, Compound 1, or a pharmaceutically acceptable salt thereof, is orally administered to a cancer patient who has been treated with another anti-cancer drug. In another embodiment, Compound 1, or a pharmaceutically acceptable salt thereof, is orally administered to a patient who has been treated with and is not responsive to another anti-cancer drug or developed resistance to such other anti-cancer compound. In another embodiment, Compound 1, or a pharmaceutically acceptable salt thereof, is orally administered to a patient who has been treated with another anti-cancer drug and is refractory to the other anti-cancer drug. Compound 1, or pharmaceutically acceptable salt thereof, may be used in orally treating cancer in a patient who is not responsive or is resistant to any other anti-cancer agent. Examples of such other anti-cancer agent may include alkylating agents, antimitotic agents, topoisomerase I inhibitors, topoisomerase II inhibitors, RNA/DNA antimetabolites, EGFR inhibitors, angiogenesis inhibitors, tubulin inhibitors, proteosome inhibitors, etc.

Pharmaceutical compositions useful within the scope of the methods of the invention include all orally useful compositions of Compound 1, or a pharmaceutically acceptable salt thereof, disclosed in International Patent Application No. PCT/US09/50036, filed Jul. 9, 2009, which is incorporated by reference herein in its entirety. Compound 1, or a pharmaceutically acceptable salt thereof, is contained in an amount that is effective to achieve its intended purpose. While individual needs vary, determination of optimal ranges of effective amounts of each component is within the skill of those in the art. The oral dosage will likely be dependent upon the age, health, and weight of the recipient, kind of concurrent treatment, if any, frequency of treatment, and the nature of the effect desired.

In addition to orally administering Compound 1, or a pharmaceutically acceptable salt thereof, as a raw chemical, such compounds of the invention may be administered as part of a pharmaceutical preparation containing suitable pharmaceutically acceptable carriers comprising excipients and auxiliaries, which facilitate processing of the compound into preparations or dosage forms that may be used pharmaceutically. Preferably, the oral preparations, such as tablets, dragees, and capsules, contain from approximately 0.01 to 99 percent, preferably from approximately 0.25 to 75 percent of active compound, together with the excipient(s).

Also included within the scope of the present invention is the use of the non-toxic pharmaceutically acceptable salts of Compound 1. Examples of pharmaceutically acceptable addition salts include inorganic and organic acid addition salts, and inorganic and organic base addition salts. Acid addition salts are formed by mixing a solution of the compound of the present invention with a solution of a pharmaceutically acceptable non-toxic acid. Basic salts are formed by mixing a solution of the compound of the present invention with a solution of a pharmaceutically acceptable non-toxic base.

The methods of the invention may be used with any animal, which may experience the beneficial effects thereof. Foremost among such animals are mammals, e.g., humans and veterinary animals, although the invention is not intended to be so limited.

The pharmaceutical preparations useful in the methods of the invention may be manufactured in a manner, which is itself known, e.g., by means of conventional mixing, granulating, dragee-making, dissolving, or lyophilizing processes. Thus, pharmaceutical preparations for oral use may be obtained by combining the active compounds with solid excipients, optionally blending and grinding the resulting mixture and processing the mixture of granules, after adding suitable auxiliaries, if desired or necessary, to obtain final blend(s) used to make tablets or dragee cores.

Suitable excipients are, in particular: fillers, cellulose preparations and/or calcium phosphates, as well as binders. If desired, disintegrating agents may be added, such as starches and also carboxymethyl-starch, cross-linked polyvinyl pyrrolidone, agar, or alginic acid or a salt thereof. Auxiliaries are, above all, flow-regulating agents and lubricants. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations are used. Dye stuffs or pigments may be added to the tablets or dragee coatings, e.g., for identification or in order to characterize combinations of active compound doses.

Other pharmaceutical preparations, which may be used orally include push-fit capsules made of gelatin, as well as soft, sealed capsules made of gelatin and a plasticizer. The push-fit capsules may contain the active compounds in the form of: granules, which may be mixed with fillers, binders, and/or lubricants, and, optionally, stabilizers. In soft capsules, the active compounds are preferably dissolved or suspended in suitable liquids, such as fatty oils, or liquid paraffin. In addition, stabilizers may be added.

The following examples are illustrative, but not limiting, of the method and compositions of the present invention. Other suitable modifications and adaptations of the variety of conditions and parameters normally encountered in clinical therapy and which are understood by those skilled in the art are within the spirit and scope of the invention.

### Example 1

### Identification of Cytotoxic Agents

A P388 murine leukemia cell line was obtained from NCI, Frederick, Md. P388 cells were cultured in RPMI-1640 supplemented with 10% fetal bovine serum, 2 mM Glutamax, 1 mM sodium pyruvate, 0.1 mM non-essential amino acids and 10 mM HEPES. Cells were grown at 37° C. in a humidified 5% CO₂ atmosphere. Exponentially growing P388 cells were plated at 5,000 cells/well in a 96-well flat-bottomed microtiter plate (Corning, Costar #3595, Lowell, Mass.). Twenty-four hours later, test compound was added to cells at final concentrations of 100 nM, 33.3 nM, 11.1 nM, 3.7 nM, 1.23 nM, 0.4 nM and 0.13 nM. Cellular viability was determined 72 hours later by measuring intracellular ATP with ATP-Lite assay system. The effect of compounds on cell viability was calculated by comparing the ATP levels of cells exposed to test compound with those of cells exposed to DMSO. A semi-log plot of relative ATP levels versus compound concentration was used to calculate the compound concentration required to inhibit growth by 50% (IC50). Data was analyzed by Prism software (GraphPad; San Diego, Calif.) by fitting it to a sigmoidal dose response curve.

A similar procedure was followed with six other cell lines and the results are shown in Table 1: HT-29, HCT-116, A549, OVCAR-3, MIAPaCa-2, and B16F1.

TABLE 1

| | | 72 Hour Cytotoxicity |
|---|---|---|
| Cell Line | Tumor | (IC50, nM ± SD) |
| HT-29 | Colorectal | 29.0 ± 2.0 |
| HCT-116 | Colorectal Carcinoma | 7.6 ± 1.9 |
| P388 | Lymphoma | 6.1 ± 2.2 |
| A549 | Lung | 54.0 |
| OVCAR-3 | Ovarian | 29.0 ± 20.1 |
| MIAPaCa-2 | Pancreatic | 56.0 |
| B16F1 | Melanoma | 37.0 ± 0.0 |

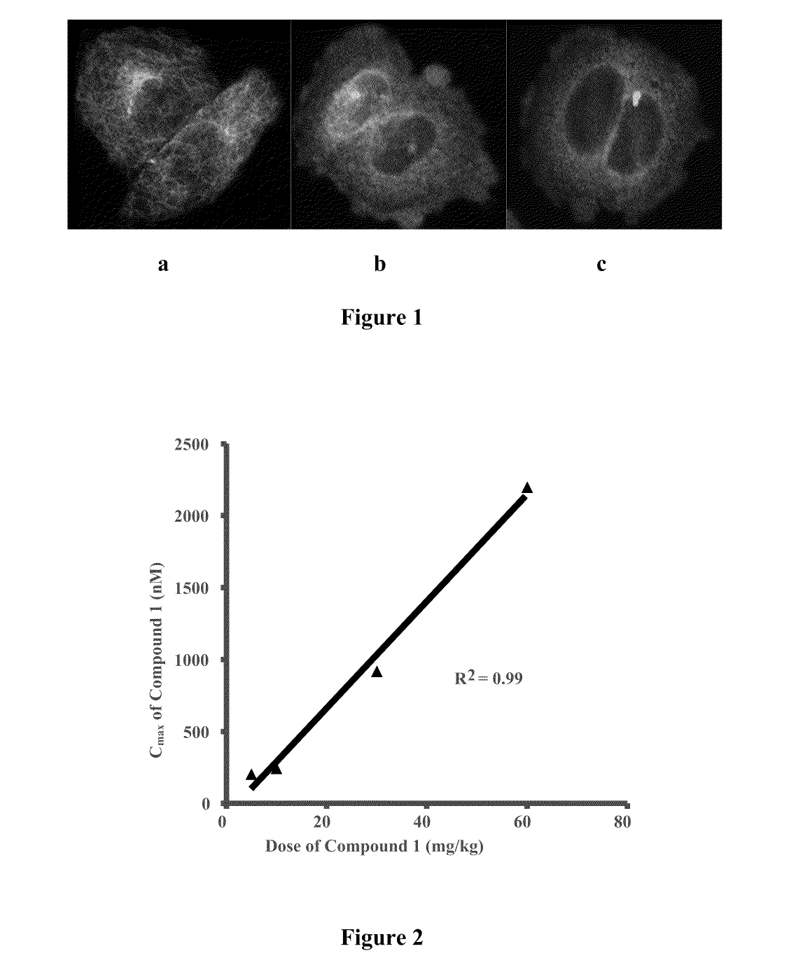

FIG. 1 depicts the extent of microtubule formation in A549 cells treated with Compound 1. The cells in panel “a” were treated with 100 nM concentration of Compound 1. The cells in panel “b” were treated with 1 μM concentration of Compound 1. The cells in panel “c” were treated with 10 μM concentration of Compound 1. It can be seen that Compound 1 inhibits microtubule formation in the A549 cells.

Accordingly, Compound 1 was identified as cytotoxic agent and is thus useful in treating the various diseases and disorders responsive to cytotoxic agents.

### Example 2

### Multidrug Resistant Cell Assays

Cytotoxicity of Compound 1 in multidrug resistant cells was determined by administering the compound to cell lines that overexpress the multidrug resistance pump MDR-1 and determining the viability of the cell lines. P388/ADR cell lines are known to overexpress the multidrug resistance pump MDR-1 (also known as P-glycoprotein-1; Pgp-1).

P388/ADR cell lines were obtained from American Type Culture Collection (Manassas, Va.) and maintained in RPMI-1640 media supplemented with 10% FCS, 10 units/ml penicillin and streptomycin, 2 mM Glutamax and 1 mM sodium pyruvate (Invitrogen Corporation, Carlsbad, Calif.). For compound testing, cells were plated in 96 well dishes at a concentration of 1.5×10⁴ cells/well. Cells were allowed to adhere to the plate overnight and then incubated with the compound at final concentrations ranging from 0.13 nM to 10 uM for 72 hours. Cell viability was then assessed using the ATP-lite reagent (Perkin Elmer, Foster City, Calif.). Plates were read on a Wallac Topcount luminescence reader (Perkin Elmer, Foster City, Calif.) and the results graphed in Prism software (Graphpad Software, Inc., San Diego, Calif.). Non-linear regression with variable slope analysis was performed to obtain IC50 concentration values.

A similar procedure was followed with the P388 cell line and the following cell lines that express multi-drug resistance ABC transporters: MCF-7, NCI/ADR-Res (MDR-1), MCF-7/MX (BCRP-1), and MCF-7/VP (MRP-1). Additionally, the cell lines were treated with other chemotherapeutics known to be substrates for ABC transporters (vinblastine, docetaxel, epirubicin, and CPT11). The results are shown in Table 2.

TABLE 2
Various MDR Cell Line Cytotoxicity IC₅₀, nM

| | Com- | | | | |
|---|---|---|---|---|---|
| | pound 1 | Vinblastine | Docetaxel | Epirubicin | CPT11 |
| P388 | 18.0 | 0.5 | 3.3 | | |
| P388/ADR | 27.0 | 8.3 | 200.0 | | |
| (MDR-1) | | | | | |
| MCF-7 | 27.0 | 1.4 | 13.0 | 160.0 | 20.0 |
| NCI/ADR-Res | 13.0 | 100.0 | 900.0 | | |
| (MDR-1) | | | | | |
| MCF-7/MX | 9.0 | | | 400.0 | 15.0 |
| (BCRP-1) | | | | | |
| MCF-7/VP | 13.0 | | | 2100.0 | |
| (MRP-1) | | | | | |

Accordingly, (2-aminomethylquinazolin-4-yl)-(4-methoxyphenyl)methylamine) (“Compound 1”) was identified as a cytotoxic agent in multidrug resistant cells and is thus useful in treating the various diseases and disorders discussed above in drug resistant cancer patients.

### Example 3

### Pharmacokinetic Studies

Animals were dosed with approximately 2.5 mg/kg i.v. in the tail vein or 5, 10, 30, or 60 mg/kg p.o. in fed and 60 mg/kg p.o. in a fasted state. Blood samples and whole brains were collected from five mice at each of the nine collection time points (approximately 0.05, 0.25, 1.0, 2.0, 4.0, 8.0, 12.0 and 24.0 hours) after administration of the dose. Plasma was collected from blood samples, and whole brain sample were homoginized. Both tissues were analyzed for the concentration of Compound 1. Pharmacokinetic parameters were estimated by non-compartmental analysis using WinNonlin.

FIG. 2 depicts the oral dose proportionality of Compound 1 to plasma Cₘₐₓ. Dose proportional increases in Cₘₐₓ were observed in mice dosed orally with Compound 1 up to at least 60 mg/kg.

Table 3 lists the pharmacokinetics of Compound 1 in mice dosed per os (p.o.) and intravenous (i.v.). No significant differences in pharmacokinetics were observed between fed and fasted animals. Compound 1 appears almost completely bioavailable after oral dosing, unlike vinca alkaloids, such as, for example, vinblastine, vincristine and vinorelbine.

TABLE 3

| | Plasma-PK parameters | | | | | | | | | | |
|---|---|---|---|---|---|---|---|---|---|---|---|
| Sub- | | Route of | Dose | t½ | Tmax | Cmax | Cmax | AUC(0-inf) | CL | V | |
| ject | Fast Status | Admin | (mg/kg) | (hr) | (hr) | (ng/mL) | (μM) | (hr*ng/mL) | (mL/hr/kg) | (mL/kg) | % F |
| 1 | Not Fasted | PO | 60 | 10.8 | 0.25 | 1120 | 3.8 | 10746 | 5583 | 86914 | 96.0 |
| 2 | Not Fasted | IV | 2.5 | 6.2 | 0.05 | 513 | 1.7 | 467 | 5358 | 47759 | |
| 3 | Fasted | PO | 60 | 18.3 | 1.00 | 1090 | 3.7 | 12855 | 4668 | 123424 | 114.8 |

| Sub- | Brain-PK parameters | | Dose | t½ | Tmax | Cmax | Cmax | AUC(0-inf) | CL | V |
|---|---|---|---|---|---|---|---|---|---|---|
| ject | Fast Status | ROA | (mg/kg) | (hr) | (hr) | (ng/mL) | (μM) | (hr*ng/mL) | (mL/hr/kg) | (mL/kg) |
| 1 | Not Fasted | PO | 60 | 9.0 | 2.00 | 6030 | 20.4 | 77290 | 776 | 10109 |

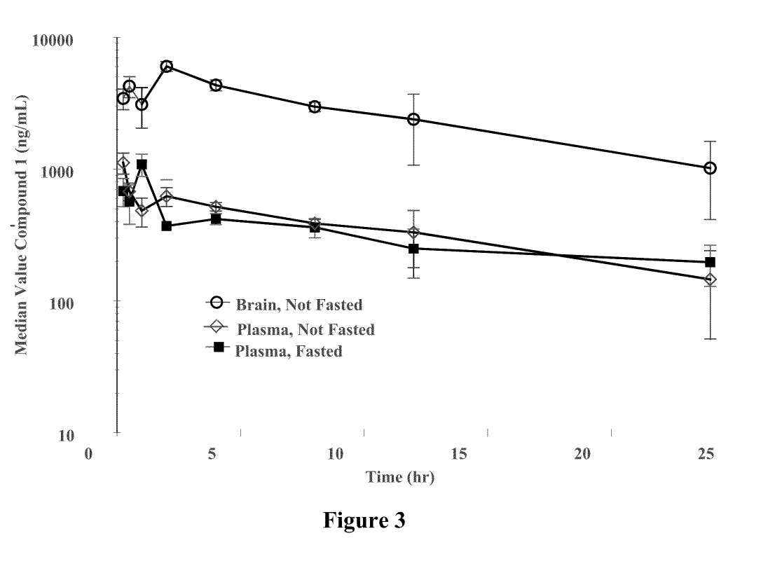

FIG. 3 depicts the plasma and brain pharmacokinetic profile in non-fasted female Swiss Webster mice and the plasma pharmacokinetic profile in fasted Swiss Webster mice resulting from a single 60 mg/kg p.o. dose. Brain concentrations of Compound 1 were approximately 7-fold higher than plasma levels (AUC_{0-∞}).

### Example 5

### Allograft Studies

Female Crl:Nu/Nu-nuBR mice (Charles River Labs, Wilmington, Mass.) were implanted subcutaneously with 10⁶ B16-F0 cells in the right flank. Animals were housed by groups in Positive Individual Ventilation cages in flat-bottom cages with no more than ten mice per cage. Environmental controls were set to maintain a temperature between 65 and 75° F. with a relative humidity of 30-70% in a 12:12 hour light:dark cycle. Animals were fed and watered ad libitum. Tumors were allowed to grow to approximately 100 mm³ and then mice were placed into test groups (N=10). Animals were treated with vehicle, 50 mg/kg Compound 1 qd×5 p.o. or 200 mg/kg Compound 1 qwk×2 p.o. The mice were observed daily for mortality and signs of toxicity. Tumor growth was monitored externally using a caliper and volumes calculated using the formula [p/6 (width²×length)], in which width represents the smaller tumor diameter. Studies were completed when the first animal achieved a tumor volume >1500 mm³. Statistical analysis of variance with unadjusted pair wise comparison was performed using SAS software (Cary, N.C.).

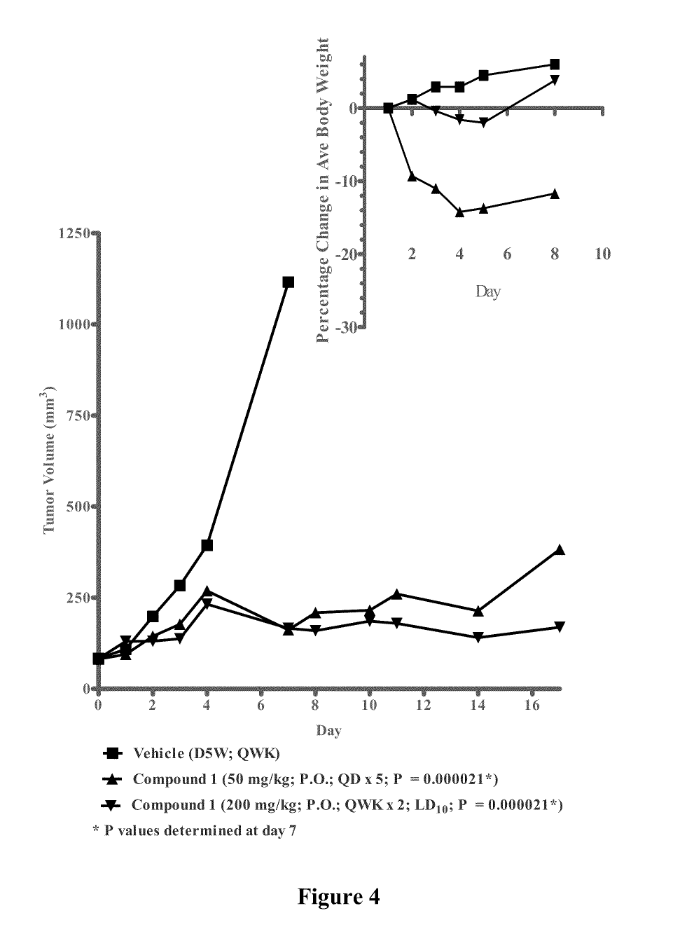

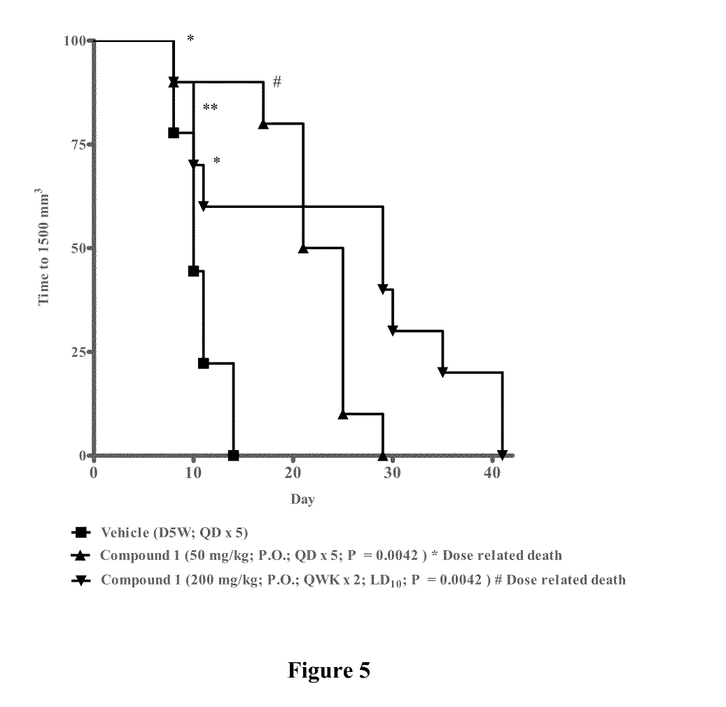

FIG. 4 depicts inhibition of B16-F0 tumor growth by Compound 1 at the different doses and regimens and also percent change in average body weight. FIG. 5 depicts time to sacrifice of the allograft mice. Statistically significant (p<0.00005) inhibition of tumor growth was observed in melanoma (B16-F0) allografts in nude mice dosed with oral Compound 1 relative to vehicle.

### Example 6

### Pharmacokinetic Study in a Primate

A single non-naïve male cynomolgus monkey was assigned to this study. The animal was fasted overnight prior to dosing, and for four hours after dosing (total fasting time did not exceed 24 hours). Compound 1 was administered once orally via a gavage tube. The gavage tube was rinsed with water and removed. Blood was removed through the femoral artery/vein into a tube containing K₃EDTA. The samples were centrifuged and the resulting plasma was separated and stored at approximately −70° C. until shipment to the analysis site. At the analysis site, the samples were stored at −20° C. until the day of analysis.

Plasma samples were analyzed for Compound 1 concentration using a LC-MS/MS method. The range of quantitation was 5-5000 ng/mL and utilized a 0.1 mL plasma sample volume. Concentration-time curves were constructed using WinNonlin (version 5.1.1). Estimated pharmacokinetic parameters are listed in Table 4 below:

TABLE 4

| Parameter | Estimate | |
|---|---|---|
| t_{1/2} | 1.9 | hr |
| Tₘₐₓ | 2.0 | hr |
| Cₘₐₓ | 166 | ng/mL |
| Tₗₐₛₜ | 12.0 | hr |
| AUC_{(0-inf)} | 535.7 | hr * ng/mL |
| CL/F_obs | 37.3 | L/hr/kg |
| Vz/F_obs | 102.0 | L/kg |

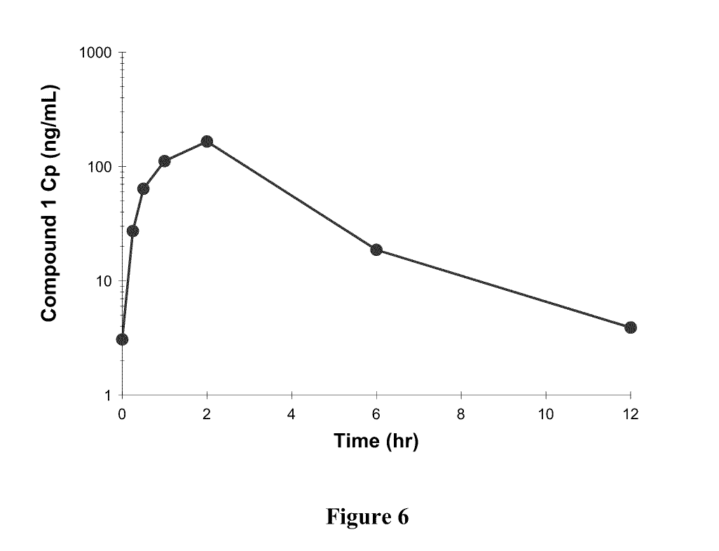

Table 5, below, lists Compound 1 plasma concentrations in the male Cynomolgus monkey per sample collection time. FIG. 6 depicts a semilog plot of Compound 1 plasma concentration in the male Cynomolgus monkey. The study shows that Compound 1 is bioavailable in a male cynomolgus monkey dosed orally at 20 mg/kg.

TABLE 5

| Post Dose Sample | Compound 1 Plasma |
|---|---|
| Time | Concentration (ng/mL) |
| 0 | 3.08* |
| 0.25 | 27.40 |
| 0.5 | 64.15 |
| 1 | 112.05 |
| 2 | 166.01 |
| 6 | 18.72 |
| 12 | 3.91* |

### Example 7

### Injection Formulation

| Components | Amount | |
|---|---|---|
| Active Compound | 5 | mg |
| PEG-400 | 5 | grams |
| TPGS | 10 | grams |
| Benzyl alcohol | 0.5 | gram |
| Ethanol | 2 | grams |
| D5W | Add to make 50 | mL |

An injection formulation of (2-aminomethylquinazolin-4-yl)-(4-methoxyphenyl)methylamine (the “Active Compound”) can be prepared according to the following method. 5 mg of the Active Compound is dissolved into a mixture of the d-α-tocopheryl polyethylene glycol 1000 succinate (TPGS), PEG-400, ethanol, and benzyl alcohol. D5W is added to make a total volume of 50 mL and the solution is mixed. The resulting solution is filtered through a 0.2 μm disposable filter unit and is stored at 25° C. Solutions of varying strengths and volumes are prepared by altering the ratio of Active Compound in the mixture or changing the total amount of the solution.

### Example 8

### Tablet Formulation

| Components | Amount | |
|---|---|---|
| Active Compound | 100.0 | mg |
| Lactose | 100.0 | mg |
| Corn Starch | 50.0 | mg |
| Hydrogenated Vegetable Oil | 10.0 | mg |
| Polyvinylpyrrolidone | 10.0 | mg |
| | 270.0 | mg |

A formulation of tablets of (2-aminomethylquinazolin-4-yl)-(4-methoxyphenyl)methylamine (the “Active Compound”) can be prepared according to the following method. 100 mg of Active Compound) is mixed with 100 mg lactose. A suitable amount of water for drying is added and the mixture is dried. The mixture is then blended with 50 mg of corn starch, 10 mg hydrogenated vegetable oil, and 10 mg polyvinylpyrrolidinone. The resulting granules are compressed into tablets. Tablets of varying strengths are prepared by altering the ratio of Active Compound in the mixture or changing the total weight of the tablet.

### Example 9

### Capsule Formulation

| Components | Amount | |
|---|---|---|
| Active Compound | 100.0 | mg |
| Microcrystalline Cellulose | 200.0 | mg |
| Corn Starch | 100.0 | mg |
| Magnesium Stearate | 400.0 | mg |
| | 800.0 | mg |

A formulation of capsules containing 100.0 mg of (2-aminomethylquinazolin-4-yl)-(4-methoxyphenyl)methylamine (the “Active Compound”) can be prepared according to the following method. 100 mg of Active Compound is mixed with 200 mg of microcrystalline cellulose and 100 mg of corn starch. 400 mg of magnesium stearate is then blended into the mixture and the resulting blend is encapsulated into a gelatin capsule. Doses of varying strengths can be prepared by altering the ratio of the Active Compound to pharmaceutically acceptable carriers or changing the size of the capsule.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be understood that certain changes and modifications may be practiced within the scope of the appended claims.

What is claimed is:

1. A method of treating or ameliorating neoplasm or cancer, said method comprising orally treating a warm-blooded animal, in need of such treatment, with an effective amount of the compound (2-aminomethylquinazolin-4-yl)-(4-methoxyphenyl)methylamine, or a pharmaceutically acceptable salt thereof.

2. The method of claim 1, further comprising administering at least one cancer chemotherapeutic agent other than said compound, or a pharmaceutically acceptable salt of said at least one cancer chemotherapeutic agent.

3. The method of claim 2, wherein said at least one cancer chemotherapeutic agent is selected from alkylating agents, antimitotic agents, topoisomerase I inhibitors, topoisomerase II inhibitors, RNA/DNA antimetabolites, DNA antimetabolites, EGFR inhibitors, proteosome inhibitors, antibodies, and combinations thereof.

4. The method of claim 1, further comprising administering radiation therapy at the same time or at a different time as treating with said compound.

5. The method of claim 1, wherein treating said warm-blooded animal with cancer further comprises surgically removing said cancer and then administering an effective amount of said compound to said warm-blooded animal.

6. The method of claim 1, wherein said cancer comprises a drug-resistant cancer.

7. The method of claim 1, wherein said cancer comprises a primary cancer.

8. The method of claim 1, wherein said cancer comprises a metastatic cancer.

9. The method of claim 1, wherein said warm-blooded animal is a human.

10. The method of claim 1, wherein said therapeutically effective amount is an amount sufficient to provide in the animal a plasma Cₘₐₓ of about 1 ng/mL to about 4,000 ng/mL, or an amount of a pharmaceutically acceptable salt of (2-aminomethylquinazolin-4-yl)-(4-methoxyphenyl)methylamine sufficient to achieve an equimolar concentration in the plasma of the animal.

11. The method of claim 10, wherein said plasma Cₘₐₓ is between about 150 ng/mL and about 1,100 ng/mL.

12. The method of claim 1, wherein said therapeutically effective amount is an amount sufficient to provide in the animal a plasma AUC_{(0 to ∞)} of about 1 hr*ng/mL to about 30,000 hr*ng/mL, or an amount of a pharmaceutically acceptable salt of (2-aminomethylquinazolin-4-yl)-(4-methoxyphenyl)methylamine sufficient to achieve an equimolar concentration in the plasma of the animal.

13. The method of claim 12, wherein said plasma AUC_{(0 to ∞)} is between about 8,000 hr*ng/mL to about 30,000 hr*ng/mL.

14. The method of claim 1, wherein said therapeutically effective amount is an amount sufficient to provide in the animal a brain tissue AUC_{(0 to ∞)} of about 1 hr*ng/mL to about 210,000 hr*ng/mL, or an amount of a pharmaceutically acceptable salt of (2-aminomethylquinazolin-4-yl)-(4-methoxyphenyl)methylamine sufficient to achieve an equimolar concentration in the brain tissue of the animal.

15. The method of claim 14, wherein said brain tissue AUC_{(0 to ∞)} is between about 56,000 hr*ng/mL to about 210,000 hr*ng/mL.

16. The method of claim 1, wherein said therapeutically-effective amount is about 20 mg to about 200 mg, or an equimolar amount of a pharmaceutically acceptable salt of (2-aminomethylquinazolin-4-yl)-(4-methoxyphenyl)methylamine, per kilogram of body weight of said animal.

17. The method of claim 16, wherein said therapeutically-effective amount is about 50 mg to about 200 mg, or an equimolar amount of a pharmaceutically acceptable salt of (2-aminomethylquinazolin-4-yl)-(4-methoxyphenyl)methylamine, per kilogram of body weight of said animal.

18. A method of treating an individual having cancer, said method comprising treating said individual with about 20 mg to about 200 mg of (2-aminomethylquinazolin-4-yl)-(4-methoxyphenyl)methylamine, or an equimolar amount of a pharmaceutically acceptable salt thereof, per kilogram of body weight of said individual.

19. The method of claim 18, wherein treating said individual comprises orally administering (2-aminomethylquinazolin-4-yl)-(4-methoxyphenyl)methylamine, or an equimolar amount of a pharmaceutically acceptable salt thereof, to said individual.

20. The method of claim 18, comprising treating said individual with about 50 mg to about 200 mg of (2-aminomethylquinazolin-4-yl)-(4-methoxyphenyl)methylamine, or an equimolar amount of a pharmaceutically acceptable salt thereof, per kilogram of body weight of said individual.
